(19) United States
(12) Patent Application Publication
(10) Pub. No.: US 2024/0105325 A1
(43) Pub. Date: Mar. 28, 2024

(54) REMOTE RESCUE

(71) Applicant: Jay Harary, Linden, NJ (US)

(72) Inventor: Jay Harary, Linden, NJ (US)

(21) Appl. No.: 17/952,384

(22) Filed: Sep. 26, 2022

(51) Int. Cl. G16H 40/40; G06Q 10/00

(52) U.S. Cl. CPC G16H 40/40; G06Q 10/20

(57) ABSTRACT

Methods for remote repair of medical devices are described. A prompt for information on how to repair a medical device associated with a unique identifier is received from a computing device associated with a user. Information associated with the medical device is identified from the unique identifier. The information includes a qualified user to engage in a remote video session with the user, a location where the medical device can be ordered from, instructions or information on how to install the medical device, and/or instructions or information on how to repair the medical device. The information is then transmitted to the user.

### FIELD OF THE EMBODIMENTS

The field of the invention and its embodiments relate to methods for remote repair of devices. In particular, the present invention and its embodiments provide methods for remote repair of medical devices.

### BACKGROUND OF THE EMBODIMENTS

The medical device industry innovates aggressively and continues to grows strongly. In 2017, the United States covered approximately 40 percent of the global medical device market, and by 2023, the U.S. medical device market is expected to grow to $208 billion. As of 2019, the top three global medical device manufacturing companies include Medtronic, Johnson & Johnson, and GE Healthcare, grossing $70 billion.

The continuous growth in the medical market is enabled by the underlying demographics and by the innovation and expansion of medical devices' clinical applications and effectiveness. Companies continue to innovate wirelessly interconnected devices, software as a medical device, cloud-based artificial intelligence, and cost-effective customization enabled by 3-D printing, among other technologies.

However, medical device innovation is expensive due to financial constraints, such as the costs associated with such devices. For example, particle accelerators may cost more than $100 million, a computed tomography (CT) scanning machine may cost approximately $2.7 million, a magnetic resonance imaging (Mill) machine and a robotic surgical machine may each cost approximately $2.3 million, and a three-dimensional (3D) mammography machine may cost approximately $760,000.

Medical device innovation is additionally expensive due to regulatory challenges, sociological hurdles, and potential litigation costs. As such, many companies are looking towards implementing medical device refurbishment schemes. Such refurbishment scheme works to restore the medical device to a condition of safety and effectiveness that is comparable to when the medical device was new. This may include reconditioning, repairing, and/or installing certain software/hardware updates that do not change the intended use of the original medical device. Such refurbishment may also include replacing worn parts of the medical device. Despite the need for such refurbishment systems, no solution in the technical field currently provides a convenient and easy system. Thus, a need exists for a user-friendly and convenient remote medical device refurbishment system.

### REVIEW OF RELATED TECHNOLOGY

JP 4,854,120 B2 describes a system for performing maintenance of a medical device in a hospital according to a maintenance contract or the like. In particular, the medical device is remotely monitored and repaired from a service center.

JP 2002/236758 A and JP 2002/233504 A relate to methods to order parts for a medical device installed in a hospital and repair an abnormality in the medical device as early as possible. State data of the medical device is collected and it is determined whether or not an abnormality has occurred in the medical device based on the collected state data. If it is determined that the abnormality has occurred, a service center will be notified. The service center then contacts a service station to repair the medical device. The service station may order parts for the medical device and may transmit them to the hospital.

U.S. Published Patent Application No. 2007/0129609 A1 describes a support network of a medical institution that provides the latest medical technology for a terminal medical institution. The support network includes terminal equipment having a diagnostic examination instrument essentially arranged in the terminal medical institutions; a communication network for transmitting diagnosis and examination data of the terminal equipment; and a complete medical institution for analyzing and processing the diagnosis and examination data transmitted and for transmitting the processed result to the terminal medical institution via the communication network.

U.S. Published Patent Application No. 2006/0214777 A1 and DE 102005013852 A1 describe a method for remote maintenance of a medical device. The method includes providing a medical device; connecting the medical device to a power line for transmitting data via the power line, the medical device comprising a control and monitoring device for operating the medical device; providing a computer at a remote location relative to the medical device; connecting the remote computer to the power line; and transmitting the data between the control and monitoring device and the computer via the power line using a data transmission device configured to transmit data over power lines. The data contains exclusively control and/or monitoring-relevant data for the medical device. The data can, for example, be control data for operating the medical device. The data can also be software data, hardware data, error data or maintenance data. The transmission of control and/or monitoring-relevant data enables repair and maintenance intervals to be defined, as well as remote maintenance of the medical device to be performed. Faults and errors can be detected at an early stage.

U.S. Published Patent Application No. 2014/0204190 A1 describes methods and systems for providing real-time guidance for a medical procedure with a device.

U.S. Published Patent Application No. 2006/0026035 A1 describes a computer-aided interactive medical management information and control method for assessing, diagnosing, planning, and/or performing a treatment on a patient by a health care provider.

Various repair methods and systems are known in the art. However, their means of operation are substantially different from the present disclosure, as the other inventions fail to solve all the problems taught by the present disclosure. The present invention and its embodiments provide methods for remote repair of devices. In particular, the present invention and its embodiments provide methods for remote repair of medical devices.

### SUMMARY OF THE EMBODIMENTS

The present invention and its embodiments provide methods for remote repair of devices. In particular, the present invention and its embodiments provide methods for remote repair of medical devices.

A first embodiment of the instant invention describes a method executed by a first computing device for remote repair of a device. The method includes receiving, from a second computing device associated with a user, a prompt for information on how to repair the device. In examples, the device is a medical device or a piece of medical equipment. In further examples, the device is a medical device and is a Class I medical device, a Class II medical device, a Class III medical device, or a Class IV medical device. It should be appreciated that the device is not limited to these examples and other devices are contemplated herein. The device is also associated with a unique identifier.

The information associated with the device includes a location where the device can be ordered from, a qualified user to engage in a remote video session with the user, instructions on how to install the device, and/or instructions on how to repair the device. The qualified user is a device repair professional. Each of the instructions on how to install the device and each of the instructions on how to repair the device include textual instructions, graphical instructions, audio instructions, and/or video instructions. The method then includes querying a memory of the first computing device to identify the information associated with the device from the unique identifier and transmitting the information associated with the device to the second computing device associated with the user.

In further examples, the prompt includes an order for the device. The method then includes querying the memory of the first computing device to identify the location where the device can be ordered from and transmitting a second order for the device to the location, where the second order includes instructions to transmit the device to the user.

In another example, the prompt further includes a request, from the user, to engage in a remote video session. In some examples, the remote video session is a real-time remote video session. In this example, the method includes transmitting a prompt to the qualified user to engage in the remote video session with the user. In response to receiving an acceptance of the request for the qualified user to engage in the remote video session with the user, the method further includes allowing the qualified user to engage in the remote session with the user.

A second embodiment of the instant invention describes a computer system. The computer system includes one or more processors, one or more memories, and one or more computer-readable hardware storage devices. The one or more computer-readable hardware storage devices contain program code executable by the one or more processors via the one or more memories to implement a method for remote repair of a medical device. The method includes: receiving, from a computing device associated with a user, a prompt for information on how to repair the medical device associated with a unique identifier. The prompt includes a request to engage in a remote video session.

The method then includes querying the one or more memories to identify the information associated with the medical device from the unique identifier. The information associated with the medical device includes: a qualified user to engage in the remote video session with the user, a location where the medical device can be ordered from, instructions on how to install the medical device, and/or instructions on how to repair the medical device. The qualified user is a medical device repair professional. Each of the instructions on how to install the device and each of the instructions on how to repair the device include textual instructions, graphical instructions, audio instructions, and/or video instructions.

The method then includes transmitting the information associated with the medical device to the computing device associated with the user and transmitting a request to the qualified user to engage in the remote video session with the user. In response to receiving an acceptance of the request for the qualified user to engage in the remote video session with the user, the method further includes allowing the qualified user to engage in the remote video session with the user.

In some examples, the prompt further includes an order for the medical device. In this scenario, the method includes: querying the one or more memories to identify the location where the medical device can be ordered from and transmitting a second order for the medical device to the location. The second order includes instructions to transmit the medical device to the user.

A third embodiment of the instant invention describes a method executed by a first computing device for remote repair of a medical device. The method includes: receiving, from a second computing device associated with a user, a prompt for information on how to repair the medical device associated with a unique identifier. The prompt includes a request to engage in a remote video session and an order for the medical device. The remote video session addresses a topic, such as a defect associated with the medical device, a problem installing the medical device, and/or a problem repairing the medical device, among other topics not explicitly listed herein.

The method also includes querying a memory of the first computing device to identify information associated with the medical device from the unique identifier. The information associated with the medical device includes: a qualified user to engage in the remote video session with the user, a location where the medical device can be ordered from, instructions on how to install the medical device, and/or instructions on how to repair the medical device. The qualified user is a medical device repair professional that meets one or more pre-determined criteria. The one or more pre-determined criteria may include a certification, a pre-determined number of years of experience, and/or a positive recommendation, among other criteria.

The method then includes transmitting the information associated with the medical device to the second computing device and transmitting another order for the medical device to the location. The other order includes instructions to transmit the medical device to the user. The method further includes transmitting a request to the qualified user to engage in the remote video session with the user.

In some examples, the method additionally includes receiving a rejection, by the qualified user, of the request to engage in the remote video session with the user. In this scenario, the method includes querying the memory of the first computing device to identify another qualified user to engage in the remote video session with the user and transmitting another request to the other qualified user to engage in the remote video session with the user. In response to receiving an acceptance of the other request from the other qualified user, the method includes allowing the other qualified user to engage in the remote video session with the user.

In general, the present invention succeeds in conferring the following benefits and objectives.

It is an object of the present invention to provide a convenient and user-friendly remote medical device refurbishment system.

It is an object of the present invention to provide a convenient and user-friendly remote medical device refurbishment system that provides information about the medical device to the user, such as: a listing of qualified users to engage in a remote video session with the user, a location where the medical device can be ordered from, instructions on how to install the medical device, and/or instructions on how to repair the medical device.

It is an object of the present invention to provide a convenient and user-friendly remote medical device refurbishment system that provides an option for the user to engage in a remote video session with a qualified user to discuss a defect associated with the medical device, a problem installing the medical device, and/or a problem repairing the medical device.

It is an object of the present invention to provide a convenient and user-friendly remote medical device refurbishment system that transmits a new or refurbished medical device to the user.

It is an object of the present invention to provide a convenient and user-friendly remote medical device refurbishment system that provides information about the medical device to the user, provides an option for the user to engage in a remote video session with a qualified user to discuss a defect associated with the medical device, and/or provides an option to transmit a new or refurbished medical device to the user.

### BRIEF DESCRIPTION OF THE DRAWINGS

FIG. 1 depicts a perspective view of a computer system configured to implement a method for remote repair of a device, in accordance with embodiments of the present invention.

FIG. 2 depicts another perspective view of a computer system configured to implement a method for remote repair of a device, in accordance with embodiments of the present invention.

FIG. 3 depicts a block diagram of a method engaged in by a computer system or a computing device for remote repair of a device, in accordance with embodiments of the present invention.

FIG. 4 is a block diagram of a computing device included within the computer system of at least FIG. 1 and FIG. 2 that is configured to implement a method for remote repair of a device, in accordance with embodiments of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

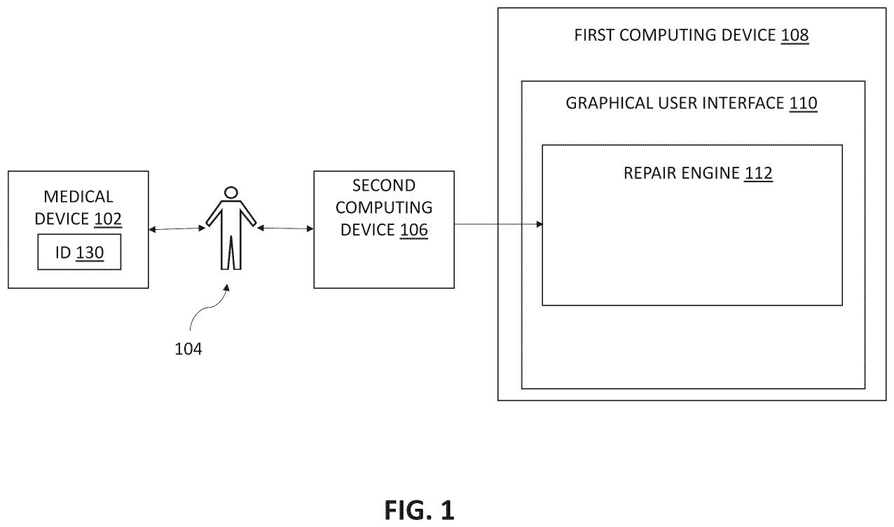

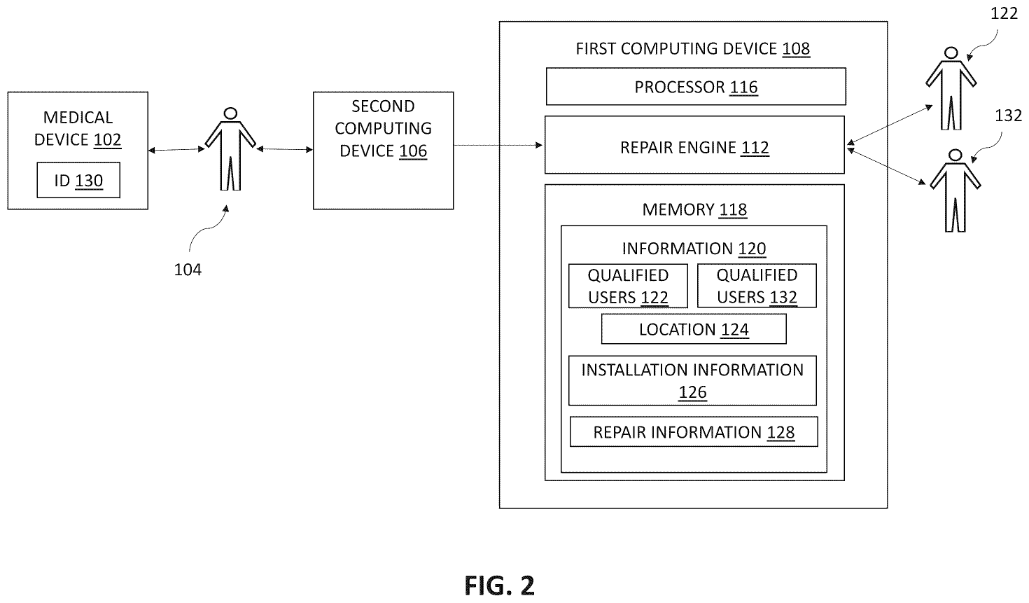

FIG. 1 and FIG. 2 depict perspective views of a computer system configured to implement a method for remote repair of a device, in accordance with embodiments of the present invention.

The computer system of FIG. 1 and FIG. 2, respectively, includes a first computing device 108 and a second computing device 106. The second computing device 106 may be associated with a first user 104. Each of the first computing device 108 and the second computing device 106 may be a computer, a laptop computer, a smartphone, and/or a tablet, among other examples not explicitly listed herein. The first computing device 108 may include a processor 116, a repair engine 112, and a memory 118.

In examples, the repair engine 112 may execute the method for remote repair of a device described infra. In examples, the device may be a medical device 102. However, the device may be another type of device. In some examples, the medical device 102 may be a Class I medical device, a Class II medical device, a Class III medical device, or a Class IV medical device. In examples, the Class I medical device comprises surgical instruments, the Class II medical device comprises contact lenses and ultrasound scanners, the Class III medical device comprises orthopedic implants and hemodialysis machines, and the Class IV medical device comprises cardiac pacemakers. It should be appreciated that the medical device is not limited to the examples or classes described herein.

In other examples, the medical device 102 may be a piece of medical equipment. Examples of medical equipment include a nebulizer, a stretcher, a crutch, an otoscope, a walker, an operating table, a glucose meter, an insulin pump, a respirator, a CT scanner, a surgical robot, an Mill scanner, and/or an anesthesia machine, among others. It should be appreciated that the pieces of medical equipment described herein are not limited to those explicitly listed.

In additional examples, the medical device 102 may be associated with a unique identifier 130. The unique identifier 130 may include a label associated with the Food and Drug Administrations' Unique Device Identification (UDI) System. In other examples, the unique identifier 130 may be a series of numbers, letters, labels, etc.

The repair engine 112 of the first computing device 108 may receive, from the second computing device 106, a prompt for information 120 on how to repair the medical device 102. The information 120 associated with the medical device 102 may include a qualified user 122 to engage in a remote video session, the qualified user 122 to engage in a textual conversation and/or a telephone call to diagnose, repair, or install the medical device 102, a location 124 where the medical device 102 can be ordered from, instructions or information on how to install 126 the medical device 102, and instructions or information on how to repair 128 the medical device 102. The qualified user 122 may be a device repair professional. In other examples, the qualified user 122 meets one or more pre-determined criteria that may include: certifications, a pre-determined number of years of experience, and/or positive recommendations.

In some examples, the instructions or information on how to install 126 the medical device 102 and the instructions or information on how to repair 128 the medical device 102 may include textual instructions, graphical instructions, audio instructions, and/or video instructions.

In response to this, the repair engine 112 of the first computing device 108 may query the memory 118 to identify the information 120 associated with the medical device 102 from the unique identifier 130. Then, the repair engine 112 of the first computing device 108 may transmit the information 120 associated with the medical device 102 to the second computing device 106 associated with the user 104.

As an illustrative example, the medical device 102 may be a component of the anesthesia machine. The prompt for the information 120 on how to repair the medical device 102 (e.g., the component of the anesthesia machine) may include a request for the instructions or the information on how to install 126 the medical device 102 (e.g., the component of the anesthesia machine). In response to this, the repair engine 112 of the first computing device 108 may query the memory 118 to identify the instructions or the information on how to install 126 the medical device 102 (e.g., the component of the anesthesia machine). Then, the repair engine 112 of the first computing device 108 may transmit the instructions or the information on how to install 126 the medical device 102 (e.g., the component of the anesthesia machine) to the second computing device 106 associated with the user 104. The instructions or the information on how to install 126 the medical device 102 (e.g., the component of the anesthesia machine) may include textual information, as well as graphical representations.

In another illustrative example, the medical device 102 may be a component of the CT scanner. The prompt for the information 120 on how to repair the medical device 102 (e.g., the component of the CT scanner) may additionally include an order request for the medical device 102 (e.g., the component of the CT scanner). In response to this, the repair engine 112 of the first computing device 108 may query the memory 118 to identify the location 124 where the medical device 102 (e.g., the component of the CT scanner) can be ordered from. The repair engine 112 of the first computing device 108 may then transmit a second order for the medical device 102 (e.g., the component of the CT scanner) to the location 124, where the second order includes instructions to transmit the medical device 102 (e.g., the component of the CT scanner) to the user 104.

In another illustrative example, the medical device 102 may include a component of the MRI scanner. The prompt for the information 120 on how to repair the medical device 102 (e.g., the component of the MM scanner) may additionally include a request to engage in a remote video session. The remote video session may address a topic including: a defect associated with the medical device 102 (e.g., the component of the Mill scanner), a problem installing the medical device 102 (e.g., the component of the MM scanner), and/or a problem repairing the medical device 102 (e.g., the component of the MM scanner), among other topics not explicitly listed herein. In response to this, the repair engine 112 of the first computing device 108 may transmit the request from the user 104 to engage in the remote video session to the qualified user 122. The repair engine 112 of the first computing device 108 may, in response to receiving acceptance of the request for the qualified user 122 to engage in the remote video session with the user 104, allow the qualified user 122 to engage in the remote video session with the user 104. In some examples, the remote video session is a real-time remote video session. It should be appreciated that the remote session described herein may also be another type of real-time remote session between the qualified user 122 and the user 104 that includes textual conversation, the sharing of files, the sharing of images, the sharing of videos, etc.

In a further illustrative example, the prompt for the information 120 on how to repair the medical device 102 may additionally include the request to engage in the remote video session. In response to this, the repair engine 112 of the first computing device 108 may transmit the request from the user 104 to engage in the remote video session to the qualified user 122. The repair engine 112 of the first computing device 108 may then receive a rejection of the request for the qualified user 122 to engage in the remote video session. The repair engine 112 of the first computing device 108 may then query the memory 118 to identify another qualified user 132 and may transmit another request to the other qualified user 132 to engage in the remote video session with the user 104. In response to receiving an acceptance of the other request from the other qualified user 132, the repair engine 112 of the first computing device 108 may allow the other qualified user 132 to engage in the remote video session with the user 104.

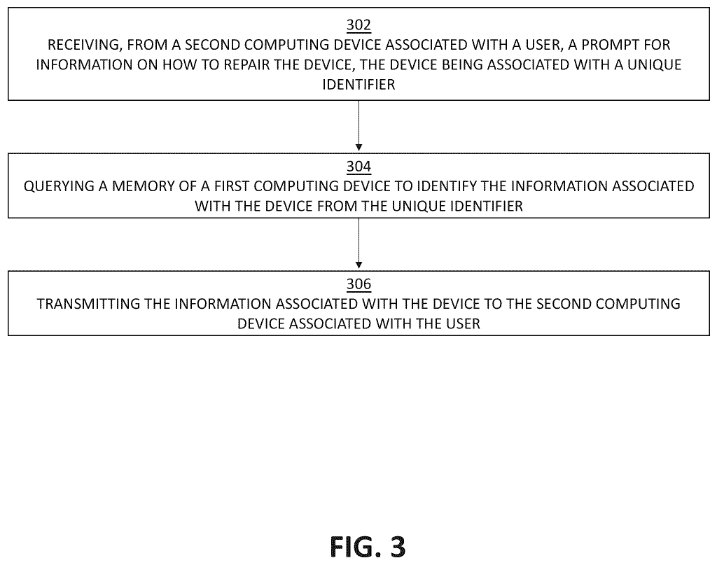

FIG. 3 depicts a block diagram of a method engaged in by a computer system or a computing device for remote repair of a device, in accordance with embodiments of the present invention.

As shown in FIG. 3, a method for remote repair of the device (e.g., the medical device 102) may be executed by a computer system or by the repair engine 112 of the first computing device 108 of FIG. 1 and FIG. 2. The method of FIG. 3 may begin at a process step 302 that includes receiving, from the second computing device 106 associated with the user 104, a prompt for the information 120 on how to repair the medical device 102. The medical device 102 is associated with the unique identifier 130.

The process step 302 is followed by a process step 304 that includes querying the memory 118 of the first computing device 108 to identify the information 120 associated with the medical device 102. The information 120 associated with the medical device 102 includes: the location 124 where the medical device 102 can be ordered from, the instructions or information on how to install 126 the medical device 102, and/or the instructions or information on how to repair 128 the medical device 102. The process step 304 is followed by a process step 306 that includes transmitting the information 120 associated with the medical device 102 to the second computing device 106 associated with the user 104.

In some examples, the prompt at the process step 302 includes an order for the medical device 102. In this scenario, the process step 306 may be followed by a new process step (not shown) that includes transmitting a second order to the location 124 where the medical device 102 can be ordered from, where the second order includes instructions to transmit the medical device 102 to the user 104.

In another example, the prompt at the process step 302 includes a request to engage in a session with the qualified user 122 to diagnose, repair, or install the medical device 102. The session may be a remote video session, a textual conversation, and/or a telephone call. In this scenario, the process step 306 may be followed by the following new process steps (not shown) that include: transmitting a prompt to the qualified user 122 to engage in the session with the user 104, receiving an acceptance of the request for the qualified user 122 to engage in the session with the user 104, and allowing the qualified user 122 to engage in the session with the user 104.

In a further example, the prompt at the process step 302 includes a request to engage in the session. In this scenario, the process step 306 may be followed by the following new process steps (not shown) that include: transmitting a prompt to the qualified user 122 to engage in the session with the user 104, receiving a rejection of the request for the qualified user 122 to engage in the session with the user 104, querying the memory 118 of the first computing device 108 to identify the other qualified user 132 to engage in the session with the user 104, transmitting the other request to the other qualified user 132 to engage in the session with the user 104, and in response to receiving an acceptance of the other request from the other qualified user 132, allowing the other qualified user 132 to engage in the session with the user 104.

It should be appreciated that the qualified user 122 and the other qualified user 132 are each medical device repair professionals that meet one or more pre-determined criteria that may include: certifications, a pre-determined number of years of experience, and/or positive recommendations, among other pre-determined criteria.

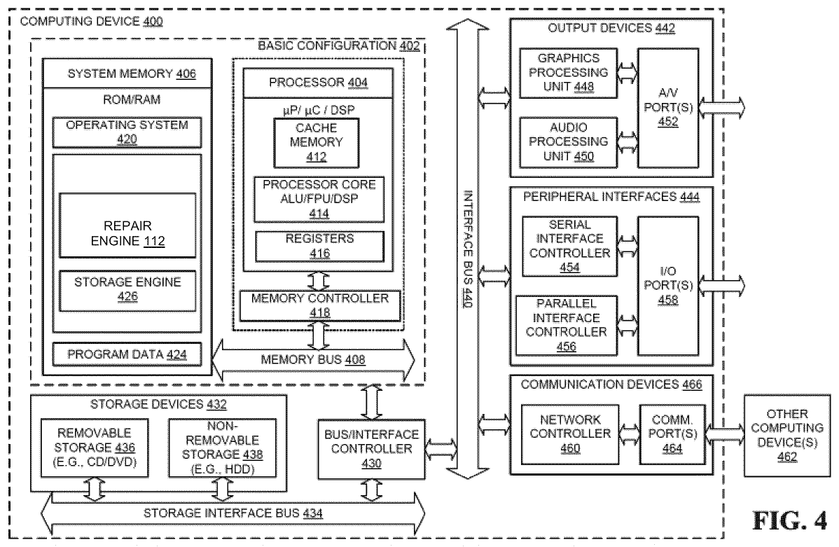

FIG. 4 is a block diagram of a computing device included within the computer system of at least FIG. 1 and FIG. 2 that is configured to implement a method for remote repair of a device, in accordance with embodiments of the present invention.

In some embodiments, the present invention may be a computer system, a method, and/or the computing device 104 (e.g., the first computing device 108 of FIG. 1 and FIG. 2) or the computing device 400 (of FIG. 4). For example, the computer system and/or the computing device 400 may be utilized to implement a method for remote repair of the medical device 102.

A basic configuration 402 of a computing device 400 is illustrated in FIG. 4 by those components within the inner dashed line. In the basic configuration 402 of the computing device 400, the computing device 400 includes a processor 404 and a system memory 406. In some examples, the computing device 400 may include one or more processors and the system memory 406. A memory bus 408 is used for communicating between the one or more processors 404 and the system memory 406.

Depending on the desired configuration, the processor 404 may be of any type, including, but not limited to, a microprocessor (μP), a microcontroller (μC), and a digital signal processor (DSP), or any combination thereof. Further, the processor 404 may include one more levels of caching, such as a level cache memory 412, a processor core 414, and registers 416, among other examples. The processor core 414 may include an arithmetic logic unit (ALU), a floating point unit (FPU), and/or a digital signal processing core (DSP Core), or any combination thereof. A memory controller 418 may be used with the processor 404, or, in some implementations, the memory controller 418 may be an internal part of the memory controller 404.

Depending on the desired configuration, the system memory 406 may be of any type, including, but not limited to, volatile memory (such as RAM), and/or non-volatile memory (such as ROM, flash memory, etc.), or any combination thereof. The system memory 406 includes an operating system 420, one or more engines, such as the repair engine 112, and program data 424. In some embodiments, the repair engine 112 may be a repair application, a repair software program, a repair service, or a repair software platform, as described infra. The repair engine 112 may be configured to implement the process steps of the methods described herein. Further, the computing device 400 may comprise a storage engine 426, which may be configured to store the information 120 used or accessed by the repair engine 112.

Moreover, the computing device 400 may have additional features or functionality, and additional interfaces to facilitate communications between the basic configuration 402 and any desired devices and interfaces. For example, a bus/interface controller 430 is used to facilitate communications between the basic configuration 402 and data storage devices 432 via a storage interface bus 434. The data storage devices 432 may be one or more removable storage devices 436, one or more non-removable storage devices 438, or a combination thereof. Examples of the one or more removable storage devices 436 and the one or more non-removable storage devices 438 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 440 facilitates communication from various interface devices (e.g., one or more output devices 442, one or more peripheral interfaces 444, and one or more communication devices 466) to the basic configuration 402 via the bus/interface controller 430. Some of the one or more output devices 442 include a graphics processing unit 448 and an audio processing unit 450, which are configured to communicate to various external devices, such as a display or speakers, via one or more A/V ports 452. The one or more peripheral interfaces 444 may include a serial interface controller 454 or a parallel interface controller 456, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 458. Further, the one or more communication devices 466 may include a network controller 460, which is arranged to facilitate communication with one or more other computing devices 462 over a network communication link via one or more communication ports 464. The one or more other computing devices 462 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 406, the one or more removable storage devices 436, and the one or more non-removable storage devices 438 are examples of the computer-readable storage media. The computer-readable storage media is a tangible device that can retain and store instructions (e.g., program code) for use by an instruction execution device (e.g., the computing device 400). Any such, computer storage media is part of the computing device 400.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 404 of a general purpose computer, special purpose computer, or other programmable data processing apparatus (e.g., the computing device 400) to produce a machine, such that the instructions, which execute via the processor 404 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 400), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

Another embodiment of the invention provides a method that performs the process steps on a subscription, advertising, and/or fee basis. That is, a service provider can offer to assist in the method steps of remote repair of a medical device. In this case, the service provider can create, maintain, and/or support, etc. a computer infrastructure that performs the process steps for one or more customers. In return, the service provider can receive payment from the customer(s) under a subscription and/or fee agreement, and/or the service provider can receive payment from the sale of advertising content to one or more third parties.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method executed by a first computing device for remote repair of a device, the method comprising:
receiving, from a second computing device associated with a user, a prompt for information on how to repair the device, the device being associated with a unique identifier;
querying a memory of the first computing device to identify the information associated with the device from the unique identifier; and
transmitting the information associated with the device to the second computing device associated with the user.

2. The method of claim 1, wherein the device is a medical device or a piece of medical equipment.

3. The method of claim 1, wherein the device is a medical device selected from the group consisting of: a Class I medical device, a Class II medical device, a Class III medical device, and a Class IV medical device.

4. The method of claim 1, wherein the information associated with the device is selected from the group consisting of: a location where the device can be ordered from, instructions on how to install the device, and instructions on how to repair the device.

5. The method of claim 4, wherein each of the instructions on how to install the device and each of the instructions on how to repair the device are selected from the group consisting of: textual instructions, graphical instructions, audio instructions, and video instructions.

6. The method of claim 1, wherein the information includes at least a location where the device can be ordered from, and wherein the prompt further includes an order for the device.

7. The method of claim 6, further comprising:
querying the memory of the first computing device to identify the location where the device can be ordered from; and
transmitting a second order for the device to the location, wherein the second order includes instructions to transmit the device to the user.

8. The method of claim 1,
wherein the prompt further includes a request to engage in a remote video session, and
wherein the information associated with the device includes a qualified user to engage in the remote video session.

9. The method of claim 8, further comprising:
transmitting a prompt to the qualified user to engage in the remote video session with the user; and
in response to receiving an acceptance of the request for the qualified user to engage in the remote video session with the user, allowing the qualified user to engage in the remote session with the user.

10. The method of claim 8, wherein the remote video session is a real-time remote video session.

11. The method of claim 8, wherein the qualified user is a device repair professional.

12. A computer system comprising one or more processors, one or more memories, and one or more computer-readable hardware storage devices, the one or more computer-readable hardware storage devices containing program code executable by the one or more processors via the one or more memories to implement a method for remote repair of a medical device, the method comprising:
receiving, from a computing device associated with a user, a prompt for information on how to repair the medical device associated with a unique identifier, wherein the prompt includes a request to engage in a remote video session;
querying the one or more memories to identify the information associated with the medical device from the unique identifier, wherein the information associated with the medical device is selected from the group consisting of: a qualified user to engage in the remote video session, a location where the medical device can be ordered from, instructions on how to install the medical device, and instructions on how to repair the medical device;
transmitting the information associated with the medical device to the computing device associated with the user;
transmitting a request to the qualified user to engage in the remote video session with the user; and
in response to receiving an acceptance of the request for the qualified user to engage in the remote video session with the user, allowing the qualified user to engage in the remote video session with the user.

13. The computer system of claim 12, wherein each of the instructions on how to install the device and each of the instructions on how to repair the device are selected from the group consisting of: textual instructions, graphical instructions, audio instructions, and video instructions.

14. The computer system of claim 12,
wherein the prompt further includes an order for the medical device, and
wherein the information associated with the medical device includes the location where the medical device can be ordered from.

15. The computer system of claim 14, wherein the method further comprises:
querying the one or more memories to identify the location where the medical device can be ordered from; and
transmitting a second order for the medical device to the location, wherein the second order includes instructions to transmit the medical device to the user.

16. The computer system of claim 12, wherein the qualified user is a medical device repair professional.

17. A method executed by a first computing device for remote repair of a medical device, the method comprising:
receiving, from a second computing device associated with a user, a prompt for information on how to repair the medical device associated with a unique identifier, wherein the prompt includes a request to engage in a remote video session and an order for the medical device;
querying a memory of the first computing device to identify information associated with the medical device from the unique identifier, wherein the information associated with the medical device is selected from the group consisting of: a qualified user to engage in the remote video session, a location where the medical device can be ordered from, instructions on how to install the medical device, and instructions on how to repair the medical device;
transmitting the information associated with the medical device to the second computing device;
transmitting another order for the medical device to the location, wherein the other order includes instructions to transmit the medical device to the user; and
transmitting a request to the qualified user to engage in the remote video session with the user.

18. The method of claim 17, further comprising:
receiving a rejection, by the qualified user, of the request to engage in the remote video session with the user;
querying the memory of the first computing device to identify another qualified user;
transmitting another request to the other qualified user to engage in the remote video session with the user; and
in response to receiving an acceptance of the other request from the other qualified user, allowing the other qualified user to engage in the remote session with the user.

19. The method of claim 17,
wherein the qualified user is a medical device repair professional that meets one or more pre-determined criteria, and
wherein the one or more pre-determined criteria include a certification, a pre-determined number of years of experience, and a positive recommendation.

20. The method of claim 17, wherein the remote video session addresses a topic selected from the group consisting of: a defect associated with the medical device, a problem installing the medical device, and a problem repairing the medical device.
